(19) United States
(12) Patent Application Publication
(10) Pub. No.: US 2022/0054019 A1
(43) Pub. Date: Feb. 24, 2022

(54) SYSTEM FOR DISTINGUISHING AN INDIVIDUAL HAVING ABNORMAL BODY TEMPERATURE

(71) Applicant: SHANY ELECTRONIC CO., LTD., Taipei (TW)

(72) Inventor: Ming-Tung TANG, Taipei (TW)

(21) Appl. No.: 17/211,510

(22) Filed: Mar. 24, 2021

(30) Foreign Application Priority Data
Aug. 24, 2020 (TW) 109128809

(51) Int. Cl. A61B 5/01; G08B 7/06; G06T 7/00; G06T 7/73; H04N 5/265; H04N 5/262; H04N 5/247; G06K 9/00; A61B 5/00; A61B 5/1171

(52) U.S. Cl. CPC A61B 5/015; G06T 2207/30201; G06T 7/0012; G06T 7/73; H04N 5/265; H04N 5/2624; H04N 5/247; G06K 9/00288; G06K 9/00268; A61B 5/0008; A61B 5/1176; A61B 5/742; A61B 5/0077; A61B 5/7405; G06T 2207/10048; G08B 7/06

(57) ABSTRACT

A system for distinguishing an individual having an abnormal body temperature includes an interface module receiving a visible light image and a thermal image, a determining module executing an image analysis process on the visible light image to recognize an individual in the visible light image and to determine position data of the individual, and a processing module obtaining a temperature value corresponding to the individual from the thermal image according to the position data. The processing module further determines whether the temperature value falls within a predetermined range of abnormal body temperature, and outputs a warning signal when it is determined that the temperature value falls within the predetermined range of abnormal body temperature.

### CROSS-REFERENCE TO RELATED APPLICATION

This application claims priority to Taiwanese Invention Patent Application No. 109128809, filed on Aug. 24, 2020.

### FIELD

The disclosure relates to a system for monitoring body temperatures of individuals, and more particularly to a system for distinguishing an individual having an abnormal body temperature.

### BACKGROUND

To forestall the spreading of diseases as attributed to gatherings, taking temperatures of people may be required as people arrive at a public place. Those people who are found to have a fever are generally refused entry into the place.

A conventional way to check people's body temperature is to use a forehead thermometer, which consumes much manpower and time. For a spacious place with lots of people, such as an airport, a train station, a hospital, a shopping center, a school, etc., a thermographic camera would be introduced for better efficiency.

The thermographic camera may detect radiant energy of individuals, convert the detected energy into temperature, and construct a thermogram to be shown on a screen. In the thermogram, each area has a specific predetermined color indicating the temperature of this area (e.g., a temperature of an individual at this area), so that an abnormally high temperature would be visibly identifiable from the thermogram. However, it is fairly difficult to recognize an individual who has a fever (i.e., a target) from among a crowd since the thermogram is presented by color lumps.

Thus, a watcher is generally assigned to watch the thermogram shown on the screen and to look around simultaneously. Once a target is shown on the screen, the watcher has to check people on the scene, and find the target from the crowd immediately. If there is no watcher or the watcher failed to locate the target on the scene, it will be a challenge to trace the target afterwards.

### SUMMARY

Therefore, an object of the disclosure is to provide a system that is used to distinguish an individual having an abnormal body temperature and that can alleviate at least one of the drawbacks of the prior art.

According to the disclosure, the system for distinguishing an individual having an abnormal body temperature includes an interface module configured to receive a visible light image and a thermal image, a determining module connected to the interface module, and a processing module connected to the interface module and the determining module.

The determining module is configured to receive the visible light image from the interface module, execute an image analysis process on the visible light image to recognize an individual in the visible light image and to determine position data of the individual on the visible light image, transmit the position data of the individual to the interface module, and output the visible light image.

The processing module is configured to receive the thermal image and the position data of the individual from the interface module, obtain a temperature value corresponding to the individual from the thermal image according to the position data, determine whether the temperature value falls within a predetermined range of abnormal body temperature, and generate and output a warning signal when it is determined that the temperature value falls within the predetermined range of abnormal body temperature.

### BRIEF DESCRIPTION OF THE DRAWINGS

Other features and advantages of the disclosure will become apparent in the following detailed description of the embodiment(s) with reference to the accompanying drawings, of which:

FIG. 1 is a block diagram of a system for distinguishing an individual having an abnormal body temperature according to an embodiment of the disclosure;

FIG. 2 is a perspective view illustrating an embodiment of a dual camera device of the system;

FIG. 3 is an exploded perspective view of the dual camera device;

FIG. 4 is a flow chart illustrating an embodiment of a method for distinguishing an individual having an abnormal body temperature according to an embodiment of the disclosure; and

FIG. 5 is a schematic view illustrating a display device displaying a thermal image and a visible light image in a split screen mode.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

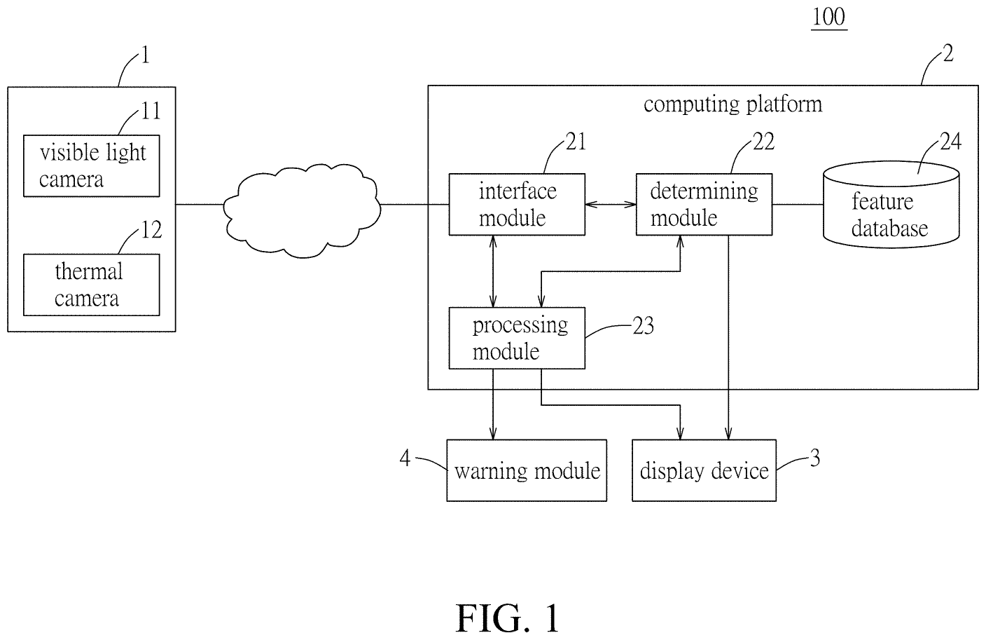

Referring to FIG. 1, one embodiment of a system 100 for distinguishing an individual having an abnormal body temperature according to this disclosure is adapted to monitor body temperatures of people and distinguish person(s) with abnormal body temperature in real time. The system 100 includes a dual camera device 1, a computing platform 2 communicating with the dual camera device 1, and a display device 3 and a warning module 4 both connected to the computing platform 2. The dual camera device 1 is normally set up at a place where people pass or enter, and includes a visible light camera 11 for capturing visible light images and a thermal camera 12 for capturing thermal images. Specifically, the visible light camera 11 and the thermal camera 12 have substantially identical fields of view, and are configured to capture images simultaneously.

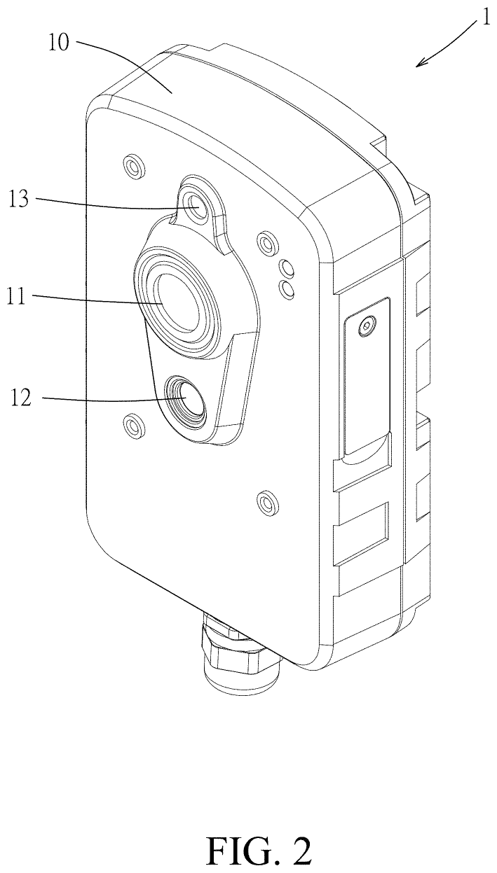

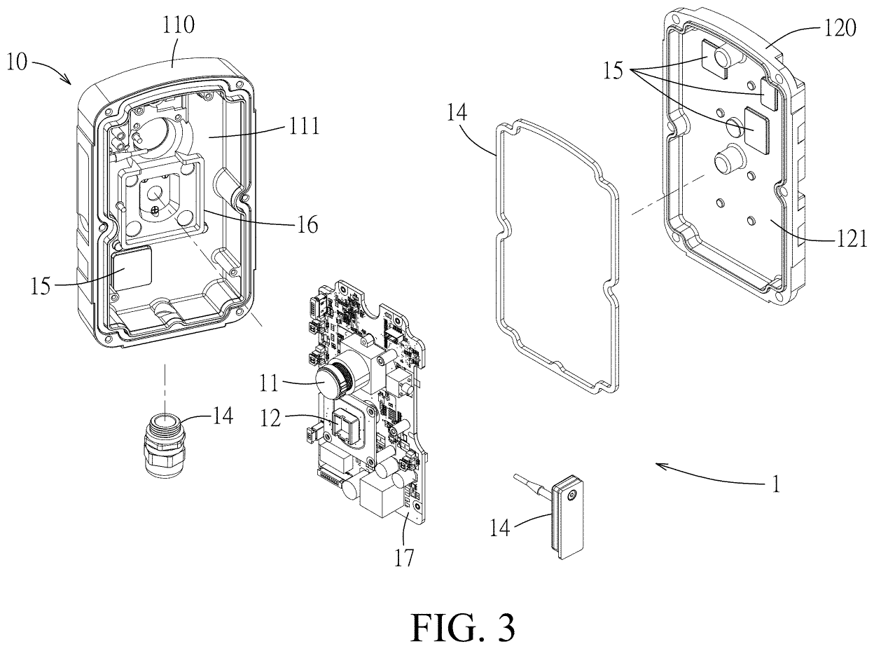

Specifically, further referring to FIGS. 2 and 3, the dual camera device 1 may further include a circuit board 17, and a case 10 for containing the circuit board 17, the visible light camera 11 and the thermal camera 12. To ensure that the visible light camera 11 can capture the visible light images with sufficient light, the dual camera device 1 may further include a light source 13, such as a light-emitting diode lamp. Furthermore, since the dual camera device 1 might be set up outdoors, the case 10 of the dual camera device 1 of this embodiment is provided with a plurality of watertight elements 14 (e.g., seal rings, plugs) placed at seams/holes of the case 10. In this embodiment, the case 10 includes a front body 110 having a first inner surface 111, and a rear body 120 being connected to the front body 110 and having a second inner surface 121 that faces the first inner surface 111. The first inner surface 111 and the second inner surface 121 cooperatively serve as an inner surface of the case 10, and define an inner space for accommodating the visible light camera 11, the thermal camera 12 and the circuit board 17. The dual camera device 1 further includes a plurality of heat spreaders 15 disposed on both the first and second inner surfaces 111, 121, and a thermal insulator 16 disposed on the first inner surface 111. The heat spreaders 15 may be heat conducting patches, and are located near heat sources, e.g., integrated circuit chips on the circular board 17, to conduct heat generated by the heat sources during operation of the dual camera device 1 to the case 10 which may be made of a material such as aluminum alloy to dissipate heat efficiently. In this embodiment, the thermal insulator 16 is in a form of a wall surrounding the thermal camera 12 so as to prevent heat in the case 10 from affecting thermal images generated by the thermal camera 12.

The computing platform 2 is a computing device, such as a server, a desktop computer, a notebook computer or a tablet, that has a storage (not shown) and a processor (not shown). The computing platform 2 may be set up at a far end and connected to the dual camera device 1 via the Internet, a local area network (LAN) or a wireless network, or may be set up at a near end and connected to the dual camera device 1 with a wire. The computing platform 2 includes an interface module 21, a determining module 22 and a processing module 23 that are built with a software development kit (SDK), and a feature database 24 stored in the storage. These modules 21-23 are application programs stored in the storage, read and executed by the processor, and connected to each other to operate cooperatively.

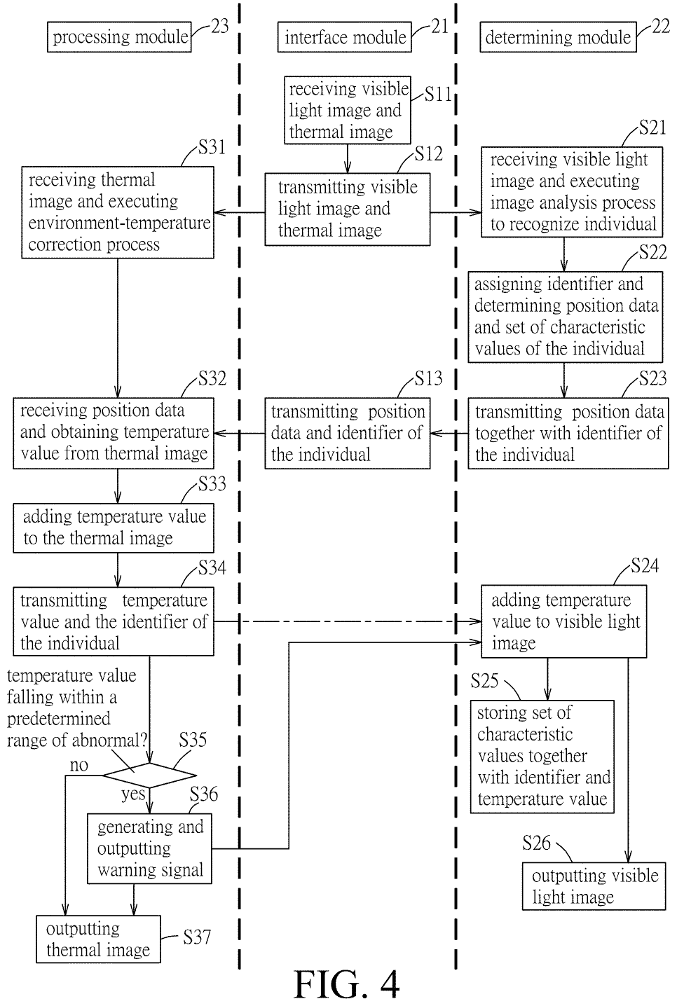

Referring to FIG. 4, the system 100 is configured to implement a method for distinguishing an individual having an abnormal body temperature and operates as follows, wherein the interface module 21 executes steps S11 to S13, the determining module 22 executes steps S21 to S25, and the processing module 23 executes steps S31 to S36.

In step S11, the interface module 21 receives visible light images from the visible light camera 11 and thermal images from the thermal camera 12. Specifically, the visible light camera 11 and the thermal camera 12 have substantially identical fields of view, and the visible light camera 11 may use streaming technologies to provide visible light images in a resolution of, for example, 2 MP (megapixels), and the thermal camera 12 may provide the thermal images of 80*60 pixels at a rate of 9 frames per second. The interface module 21 may obtain the visible light images and the thermal images at a rate of 3 frames per second, and scale or crop the visible light images and the thermal images into the same length-to-width ratio. In this way, the interface module 21 obtains a pair of images including one visible light image and one thermal image that have the same length-to-width ratio and that are captured at the same time point. Further, the rate of obtaining the pair of images may be preset by demand or may depend on the hardware efficiency of the computing platform 2. For example, at a place where people always pass fast, the rate should be increased. For the computing platform 2 having relatively greater hardware efficiency, the interface module 21 may be set to obtain a pair of images including one visible light image and one thermal image at a rate of 9 frames per second, and thus each of the thermal images captured by the thermal camera 12 will be processed. Accordingly, the computing platform 2 may perform the temperature monitoring on every individual without overlooking any.

In step S12, the interface module 21 transmits each of the visible light images received in step S11 to the determining module 22 for performing step S21, and transmits each of the thermal images received in step S11 to the processing module 23 for performing step S31.

It should be noted that the following steps are executed for each of the visible light images received by the determining module 22 and each of the thermal images received by the processing module 23.

In step S21, the determining module 22 receives the visible light image and executes an image analysis process on the visible light image to recognize at least one individual in the visible light image. In this embodiment, the determining module 22 uses face recognition technologies (e.g., using multiple predetermined face-feature templates) to detect a portion of the visible light image that matches one of the face-feature templates and deem the portion as a face (i.e., the portion of the visible light image is recognized as an individual). Then, in step S22, the determining module 22 assigns an identifier to the individual and determines position data of the individual on the visible light image.

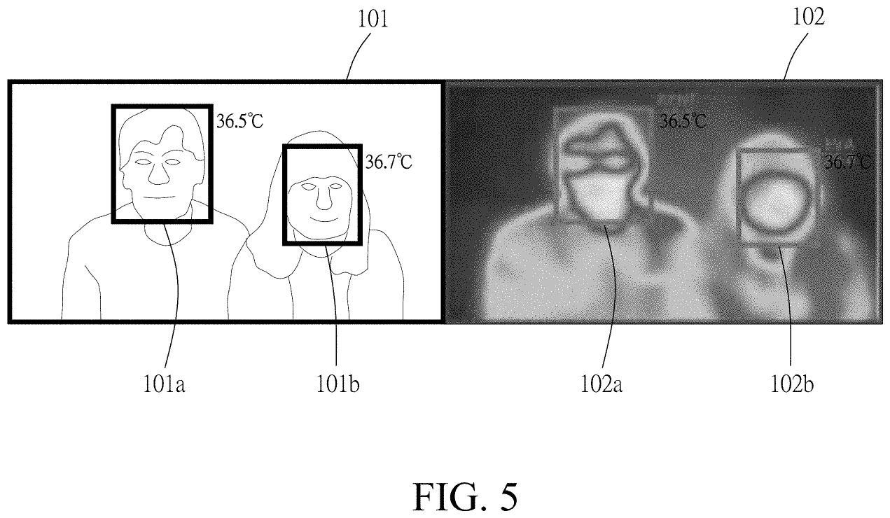

For example, the determining module 22 may detect two faces (i.e., recognize two individuals) in a visible light image 101 (see FIG. 5), and assign two identifiers to the individuals, respectively. The determining module 22 then processes the visible light image 101 by marking the individuals with two first frames 101a, 101b surrounding the faces, respectively. For each of the first frames 101a, 101b, the determining module 22 further determines two sets of coordinates for two points on the first frame 101a, 101b, and the two sets of coordinates serve as the position data. It is noted that each of the first frames 101a, 101b may be a rectangular frame, and the position data thereof may be two sets of coordinates of two nonadjacent angle points (e.g., upper-left corner and lower-right corner) of the rectangular frame. Alternatively, each of the first frames 101a, 101b may be a circular frame, and the position data thereof may include a set of coordinates of a center of the circular frame and a set of coordinates of a point on a circumference of the circular frame (or a radius of the circular frame).

It is noted that the way to recognize an individual is not limited to the face recognition technologies. Besides the face-feature templates, other features, such as features of a shoulder, may be adopted as well.

In step S23, for each individual recognized, the determining module 22 transmits the position data together with the identifier of the individual to the interface module 21. The interface module 21 then, in step S13, transmits the position data and the identifier of the individual to the processing module 23.

The processing module 23, on one hand, receives the thermal image from the interface module 21 and executes an environment-temperature correction process on the thermal image (step S31), and on the other hand, receives the position data and the identifier of the individual from the interface module 21 and reads a temperature value corresponding to the individual from the thermal image according to the position data (step S32). The environment-temperature correction process may use known temperature compensation techniques or adjust offsets using a reference blackbody with a specific temperature. Specifically, in step S32, the processing module 23 generates a second frame on the thermal image based on the position data (the two sets of coordinates), and obtains the temperature value (which has been corrected in step S31) corresponding to the individual from the thermal image by reading a highest temperature value within the second frame. For each individual, the second frame on the thermal image is identical to the first frame on the visible light image in various attributes, such as a position, a size and an orientation. In other embodiments, in order to obtain the temperature value corresponding to the individual, the processing module 23 may compute an average value of temperature values within the second frame, or read a temperature value at a specific location within the second frame, such as a center point of an upper-half part of the second frame in the case that the second frame corresponding to a human face, in which case the temperature value may serve as a forehead temperature.

Take the visible light image 101 and the thermal image 102 shown in FIG. 5 as an example, when two human faces (i.e., two individuals) are detected, the determining module 22 marks the faces respectively with two rectangular frames 101a, 101b on the visible light image 101, and determines two sets of coordinates of two nonadjacent angle points on each of the frames 101a, 101b. The processing module 23 marks two rectangular frames 102a, 102b on the thermal image 102 that correspond respectively to the frames 101a and 101b in position, and obtains two temperature values corresponding respectively to the two individuals from the frames 102a, 102b, respectively.

In step S33, for each individual, the processing module 23 adds the temperature value to the thermal image 102 near the second frame 102a, 102b. Further, in step S34, the processing module 23 transmits the temperature value together with the identifier of the individual to the determining module 22 via the interface module 21, so that the determining module 22 may add the temperature value to the visible light image 101 near the first frame 101a, 101b of the individual that corresponds to the identifier received from the processing module 23 (step S24).

The system 100 may be used at a specific site such as a school or a workplace, where specific persons such as students, teachers or staff visit regularly. Records of entry of the specific persons into the specific site and body temperatures of the specific persons would be regularly recorded for data analysis or for management purposes. In this case, the determining module 22 further analyzes the portion detected in step S21 to obtain a set of characteristic values of the individual in step S22. The set of characteristic values may be expressed by a multi-dimensional vector that is related to multiple parameters; that is to say, the characteristic values are related to the multiple parameters, respectively. Each of the parameters may represent one of human facial features, such as a distance between eyes, an angle of left eye or right eye (relative to the horizon), an aspect ratio of a forehead, etc. In step S25, the determining module 22 stores in the feature database 24 the set of characteristic values together with the identifier and the temperature value of the individual corresponding to the identifier that was received from the processing module 23. For any two similar sets of characteristic values (i.e., a product of two multi-dimensional vectors of the two sets of characteristic values higher than a preset value), the two sets of characteristic values would be deemed to correspond to the same individual.

It is noted that the processing module 23 further executes step S35 to determine whether the temperature value obtained in step S32 falls within a predetermined range of abnormal body temperature. The processing module 23 proceeds to step S36 to generate and output a warning signal when it is determined that the temperature value falls within the predetermined range of abnormal body temperature. The range of abnormal body temperature is set to, for example, 37.5 to 43° C., and an upper limit thereof (i.e., 43° C.) is used to avoid misjudgment of a fever due to the presence of hot drinks or food which may have a temperature exceeding the upper limit.

In this embodiment, the warning signal is a triggering signal. The warning module 4 may be a speaker (not shown) or a lighting device (not shown) connected to the computing platform 2, and may receive and be triggered by the warning signal to output a warning in a form of sound or flashing light for notification purposes. In other embodiments, the warning module 4 may be a communication software module (not shown) installed in the computing device, which serves as the computing platform 2, and may be triggered by the warning signal to send a message (text or image) to preset objects (such as disease prevention workers). The processing module 23 may further transmit the warning signal to the determining module 22 which then processes the visible light image 101 in response to receipt of the warning signal. The determining module 22 makes the first frame of the individual and the temperature value added near the first frame both the color red in response to receipt of the warning signal. In this way, it would be relatively easier to identify and track the individual having an abnormal body temperature. All the warning mechanisms mentioned above may be carried out cooperatively to enhance the effect of warning.

Finally, the determining module 22 outputs the visible light image 101 (step S26) and the processing module 23 outputs the thermal image 102 (step S37) to the display device 3 at the same time so as to enable the display device 3 to simultaneously display the visible light image 101 processed by and received from the determining module 22 and the thermal image 102 processed by and received from the processing module 23, in a split screen mode, as shown in FIG. 5. The visible light image 101 and the thermal image 102 displayed by the display device 3 may be recorded for review. In other embodiments, step S37 may be omitted and the display device 3 only displays the processed visible light image 101.

To sum up, since the system 100 utilizes the thermal images together with the visible light images to analyze the body temperatures of multiple persons, the system 100 can facilitate the distinguishing and identification of an individual having abnormal body temperature. Further, the system 100 can efficiently notify specific objects (e.g., disease prevention workers) of the presence of an individual who has fever via the warning mechanisms.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A system for distinguishing an individual having an abnormal body temperature, comprising:
an interface module configured to receive a visible light image and a thermal image;
a determining module connected to said interface module and configured to
receive the visible light image from said interface module,
execute an image analysis process on the visible light image to recognize an individual in the visible light image and to determine position data of the individual on the visible light image,
transmit the position data of the individual to said interface module, and
output the visible light image; and
a processing module connected to said interface module and configured to
receive the thermal image and the position data of the individual from said interface module,
obtain a temperature value corresponding to the individual from the thermal image according to the position data,
determine whether the temperature value falls within a predetermined range of abnormal body temperature, and
generate and output a warning signal when it is determined that the temperature value falls within the predetermined range of abnormal body temperature.

2. The system of claim 1, wherein said determining module is further configured to assign an identifier to the individual and transmit the identifier together with the position data of the individual to said processing module via said interface module, and said processing module is further configured to transmit the identifier and the temperature value of the individual that corresponds to the identifier to said determining module via said interface module,
wherein said determining module is further configured to process the visible light image by marking the individual with a first frame when the individual is recognized, and, after receiving the identifier and the temperature value from said processing module, to further process the visible light image by adding the temperature value to the visible light image near the first frame of the individual that corresponds to the identifier received from said processing module.

3. The system of claim 2, wherein said processing module is further configured to transmit the warning signal to said determining module, and said determining module is configured to further process the visible light image by making the first frame of the individual and the temperature value added near the first frame both color red in response to receipt of the warning signal.

4. The system of claim 3, further comprising a display device connected to said determining module for receiving the visible light image processed by said determining module, and configured to display the visible light image thus received.

5. The system of claim 2, further comprising a display device connected to said determining module for receiving the visible light image processed by said determining module, and configured to display the visible light image thus received.

6. The system of claim 5, wherein said processing module is further configured to process the thermal image by generating a second frame based on the position data of the individual on the thermal image, and adding the temperature value to the thermal image near the second frame,
wherein said display device is further connected to said processing module for receiving the thermal image processed by said processing module, and is configured to simultaneously display the thermal image processed by and received from said processing module and the visible light image processed by and received from said determining module, in a split screen mode.

7. The system of claim 1, further comprising a dual camera device connected to said interface module, and including a visible light camera and a thermal camera that have substantially identical fields of view and that are configured to capture the visible light image and the thermal image, respectively.

8. The system of claim 7, wherein said dual camera device further includes:
a case containing said visible light camera and said thermal camera, and having an inner surface; and
a thermal-insulating element being disposed on said inner surface of said case and surrounding said thermal camera.

9. The system of claim 1, further comprising a warning module connected to said processing module, and configured to receive and be triggered by the warning signal to output a warning in a form of one of sound, flashing light and a message.

10. The system of claim 1, wherein said determining module executes the image analysis process to:
use face recognition technologies to detect a portion of the visible light image that matches a predetermined face-feature so as to recognize the individual in the portion of the visible light image;
generate a first frame to surround the portion;
determine two points on the frame; and
determine two sets of coordinates respectively of the two points, wherein the two sets of coordinates serve as the position data.

11. The system of claim 10, wherein said processing module is further configured to generate a second frame on the thermal image based on the two sets of coordinates, the second frame on the thermal image being identical to the first frame on the visible light image,
wherein said processing module is configured to obtain the temperature value corresponding to the individual from the thermal image by one of reading a highest temperature value within the second frame, computing an average value of temperature values within the second frame, and reading a temperature value at a specific location within the second frame.

12. The system of claim 10, further comprising a feature database connected to said determining module,
wherein said determining module is further configured to assign an identifier to the individual and transmit the identifier together with the position data of the individual to said processing module via said interface module, and said processing module is further configured to transmit the identifier and the temperature value of the individual that corresponds to the identifier to said determining module via said interface module,
wherein, when said determining module detects the portion that matches the predetermined face-feature, said determining module further analyzes the portion to obtain a set of characteristic values,
wherein, after receiving the temperature value and the identifier from said processing module via said interface module, said determining module stores in the feature database the identifier, the temperature value received from said processing module, and the set of characteristic values of the portion having the individual that corresponds to the identifier received from said processing module.
